(19) United States
(12) Patent Application Publication
(10) Pub. No.: US 2022/0256895 A1
(43) Pub. Date: Aug. 18, 2022

(54) Method and Device for Cooking to Preserve Nutritional Value

(71) Applicants: Mihira Karnik, Chandler, AZ (US); Ajay Gawali, Chandler, AZ (US)

(72) Inventors: Mihira Karnik, Chandler, AZ (US); Ajay Gawali, Chandler, AZ (US)

(21) Appl. No.: 17/673,664

(22) Filed: Feb. 16, 2022

Related U.S. Application Data
(60) Provisional application No. 63/150,393, filed on Feb. 17, 2021.

(51) Int. Cl. A23L 5/10; G01N 33/02

(52) U.S. Cl. CPC A23L 5/13; A23V 2002/00; G01N 33/025

(57) ABSTRACT

Vegetables have high nutritional content and can bring a lot of health benefits to the consumer. Vegetables are loaded with vitamins and other micronutrients that are essential for health of an individual. Although consuming raw vegetables can offer these health benefits, many people cannot consume raw vegetables due to stomach issues. Majority of population resorts to cooking their vegetables before consuming. Cooking vegetables makes it easier to digest especially for the population with sensitive stomachs. However, cooking of vegetables results in deterioration of the nutritional content in food. Therefore, the consumer is deprived of the health benefits that the raw vegetables can bring. This invention comprises of a method and a device for cooking vegetables to conserve these vital nutrients in food by cooking the food till its optimal internal temperature. This results in degradation of cellulose that is responsible for stomach issues in people with sensitive stomach. Meanwhile the important micronutrients are not denatured and one can benefit from consumption of these ideally cooked vegetables.

This application claims the benefit of U.S. Provisional patent application No. 63/150,393 filed on Feb. 17, 2021 all of which this application is incorporated herein by reference in its entirety.

### FIELD OF THE INVENTION

The present invention is a method of cooking food and a cooking device to preserve the nutritional benefits from food.

### BACKGROUND

It is important to consume food with a higher nutritional value for better health. Raw vegetables retain their maximum nutritional value because all of their enzymes and nutrients are still intact. Unfortunately, about 15.3 million people in the United States alone suffer from indigestion when they eat raw vegetables.

One way to avoid indigestion from raw vegetables is to cook them. A drawback to cooking vegetables is that when vegetables are heated, some of the vegetable's nutritional value is lost, primarily because of the denaturation of enzymes and degradation of micronutrients. What is needed is a method of cooking that minimizes indigestion and maximizes nutritional value.

### SUMMARY OF THE INVENTION

The present invention comprises a method of cooking vegetables to preserve the nutritional content of the vegetables. This method cooks food to a point where it becomes easy to digest, while preserving the food's nutritional content. A cooking device to enable this method of cooking vegetables is presented.

Main purpose of the present invention is to provide a kind of method and system that can evaluate the internal temperature of the food as well as monitor nutrient levels in the food while cooking and give an output signal.

A cooking device can use a method of cooking wherein the food can be cooked to degrade cellulose for ease of digestion but keep the nutrients intact to get benefits from the vital nutrients in vegetables by cooking them to a predetermined level, Similarly, for meats, poultry and fish, it can cook the food to a certain degree as desired by the consumer.

There has thus been outlined, rather broadly, the more important features of the invention in order that the detailed description thereof that follows may be better understood.

The invention is not limited in its application to the details of arrangements of the components and construction of the invention set forth in the following description.

Although the invention description details the preferred embodiment, the invention is capable of other embodiments and of being practiced and carried out in various ways.

The structure, overall operation and technical characteristics of the present invention will become apparent with the detailed description and drawings of a preferred embodiment, as follows.

### BRIEF DESCRIPTION OF THE DRAWINGS

FIG. 1 is the collage of microscopic images of different vegetables cooked for different period of time.

FIG. 2. is a front perspective view of the preferred embodiment of the invention.

### DETAILED DESCRIPTION OF THE INVENTION

This invention is designed to facilitate efficient cooking method in order to preserve the nutrient composition of the vegetables. We have found a direct correlation between the internal temperature of a vegetable and the effect on nutritional content of the vegetable. Cooking vegetables is essential especially for the population who cannot digest raw food owing to a sensitive stomach.

Cell walls in plants are made of a structural carbohydrate called cellulose. Carbohydrates are one of the four classes of macromolecules and provide immediate energy. Typically ending the letters “ose,” they are made of monosaccharides that bond to form polysaccharides. Cellulose is a complex polysaccharide. It is indigestible for carnivores because they don't have the enzyme cellulase. The purpose of cellulase is to break down cellulose. Herbivores, on the other hand, have cellulase; they can consume large amounts of plants. Humans, especially those with sensitive stomachs have difficulties digesting raw vegetables and may get stomach problems such as irritable bowel syndrome, indigestion, bloating, etc. Ingesting broken down cellulose is better for sensitive stomachs. One of the methods of breaking down cellulose is to increase temperature. Intact cells when exposed to higher temperature, break down their cell walls (due to cellulose breaking). The breakdown of cell walls can be viewed under a microscope. If each individual cell can be viewed, then the cell walls are intact; therefore, the cellulose has not broken down. If each cell cannot be differentiated, then the cell walls are not intact and the cellulose has broken down.

Vegetables are rich sources of essential vitamins, minerals and disease fighting anti-oxidants. Although most vegetables can be eaten raw, most are commonly cooked either from a health perspective or for the recipe needed for consumption. Generally, preparation of vegetables is based on taste preference and very little to no attention is given to the nutrient content in these vegetables. Diet rich in vegetables can be a good source of nutrients that can protect the human body from various diseases,

During the process of cooking, as the internal temperature of the cooking pot increases, the internal temperature of the vegetable also increases. Cooking progresses for each vegetable, and there is a change in the internal temperature of the vegetable.

Table 1 shows the direct correlation between increase in the internal temperature of vegetables as cooking time increases. Table 1 shows a steady increase in the internal temperature of bell pepper, onion, broccoli, radish and zucchini with increased cooking time. As the cooking time increases, the internal temperature of the vegetables increases. The internal temperature of vegetables increases corresponding to an increase in the internal temperature of the ambient air inside the pot, and the temperature of the liquid in the pot (not shown). Initially, the temperature of the inside air of the pot is higher than the internal temperature of the vegetable by about 3-4 degree Celsius (not shown). As the cooking time increases (more than 4-6 min of cooking), the internal temperature of the cooking pot and the temperature of the liquid in the pot is the same temperature as the internal temperature of the vegetable (not shown).

Various vegetables (50 g) were placed in the cooking pot. The vegetables were placed in water (50 ml) and the temperature of the water, internal pot and vegetables were noted down, The pot was heated and the internal temperature of the vegetable as well as the water and pot was noted down at regular time intervals. The internal temperature of vegetables, pot and water was about the same in each of the cases as the heating time increased. Table 1 summarizes the change in internal temperature of the vegetable corresponding to the increase in cooking time.

TABLE 1
The effect of steaming time on internal temperature of vegetables

| Time of | | | | | |
|---|---|---|---|---|---|
| cooking | Bellpepper | Onion | Broccoli | Radish | Zucchini |
| 0 min | 18.9° C. | 19.5° C. | 18.3° C. | 16.3° C. | 19.8° C. |
| 4 min | 23.1° C. | 24.6° C. | 22.5° C. | 26.4° C. | 27.8° C. |
| 8 min | 33.7° C. | 34.6° C. | 29.2° C. | 35.1° C. | 35.3° C. |
| 12 min | 67.7° C. | 69.8° C. | 68.6° C. | 69.5° C. | 71.6° C. |
| 16 min | 79.5° C. | 71.5° C. | 79.6° C. | 72.5° C. | 78.5° C. |

The temperature of the ambient air inside the cooking pot was higher than the internal temperature of the vegetables by about 3-6° C. during the first 4-6 min of cooking. As the cooking time increased (more than 6 minutes), the temperature of most of the vegetables matched with the temperature of the ambient air inside the cooking pot.

For the purposes of this specification, we can conservatively use the temperature of the ambient air inside the cooking pot as the internal temperature of the cooking pot.

The temperature change of the cooking pot also corresponds to changes in the morphology of the vegetables such as color of the vegetable and texture of the vegetable (not shown).

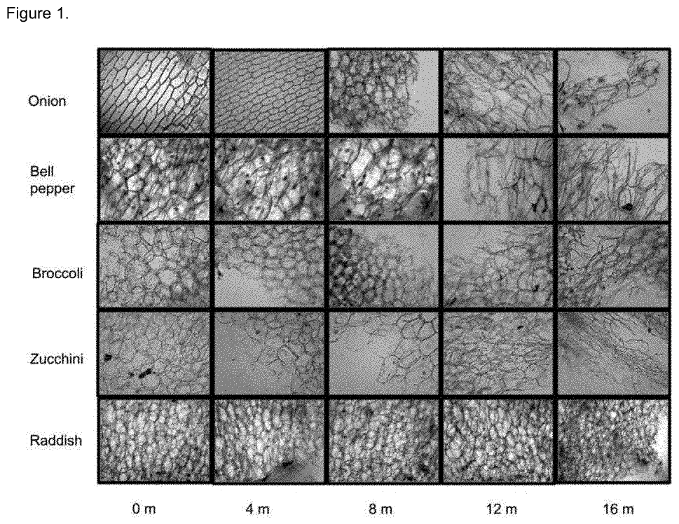

In particular, the change can be documented at a cellular level. Wherein, the cell structure changes with increasing temperature. Cell structure changes can be revealed by microscopy techniques since the cellulose present in the cell wall surrounding a cell starts breaking down. Breakdown in cellulose results in a different morphology of the cell. FIG. 1 illustrates the change in cellular morphology of different vegetables as the internal temperature of the vegetables increases. Slides of the cooked vegetables at different internal temperatures were prepared and examined under a compound microscope. Changes in morphology was documented and number of cells with broken cell walls were counted in each field of vision (not shown). A total of nine readings were taken per slide. An average of the number of broken cells were calculated per slide (not shown).

Microscopy images of cell structures of onion, bell pepper, broccoli, zucchini and radish as seen in FIG. 1 shows intact cells at room temperature (0° C.) with distinct cell walls. As internal temperature increases, the cell volume is seen to increase in size. At internal temperatures ranging between (22-35° C.), the cellular morphology shows a change in the majority of the vegetables. The change in morphology is due to the slight breakdown of cellulose and hence change in the cell wall structure. During that temperature range, about 1-10% of the cells had broken cell walls and the majority of the other cells had changed in morphology. Although most of the other cells did not have broken cell walls, the cellulose present in the cell walls had started weakening owing to the increased cell volume (turgor pressure) in the cells. This would be the ideal time to cook the vegetable since the consumers with sensitive stomachs will be able to digest the vegetables as most of the cellulose is fragile and can be easily digested.

As internal temperature of the vegetables increases (35-80° C.) after about 8 min of cooking time, bursting of the cells is observed in all the different vegetables observed under the microscope as evident in FIG. 1. This is owing to complete breaking down of the cell wall and in turn leading to opening of the cells.

One of the main reasons for consuming vegetables is that they are rich in vitamins and hence it is essential to preserve them in the cooked food. Vitamins are generally stable at body temperature and degrade as the bonds get broken. Therefore, increased temperatures will have less amounts of vitamins. The presence of vitamins can be tested with various qualitative and quantitative assays.

Another quality observed was the deterioration of micronutrients such as vitamins (for example, Vitamin B, Vitamin C) as cooking progresses. The micronutrient deterioration had a direct correlation with the increased internal temperature of the vegetables. This change in micronutrient content can be quantified using micronutrient assays.

Vitamin B has Folic acid (also known as folate or C₁₉H₁₉N₇O₆) which is also an extremely beneficial nutrient. It is a vitamin that is used to treat anemia and prevent many cardiovascular diseases. Folic acid is also considered to have a high antioxidant activity. Vegetables are natural sources of Vitamin B. Broccoli, in particular, is known to be a rich source of Vitamin B and has one of the highest content of folic acid. DPPH assay was used to quantify the amount of folic acid in broccoli.

DPPH: 1,1 Diphenyl 2-Picryl Hydrazyl is a stable (in powder form) free radical with red color which turns yellow when scavenged. In the DPPH assay, this characteristic is used to show free radical scavenging activity.

The scavenging reaction between (DPPH) and an antioxidant (HA) can be written as,

$$( DPPH ) + ( H - A ) \to DPPH - H + ( A )$$

Antioxidants react with DPPH and reduce it to DPPH-H and therefore the absorbance decreases. The degree of discoloration indicates the scavenging potential of the antioxidant compounds or extracts in terms of hydrogen donating ability.

Table 2 shows the effect of temperature on the decrease in the amount of folic acid in broccoli using a DPPH assay. Breakdown of DPPH in the presence of folk acid was quantified using a spectrophotometer at an absorbance of 515 nm wavelength (not shown). Since folic acid breaks down DPPH, denaturation of folic acid would result in intact DPPH. In this test, folic acid breaks down DPPH and changes the color of DPPH to pale yellow. If the folic acid in broccoli gets denatured by heating, there will not be a significant change in color of DPPH. Changes in the color values were quantified using a spectrophotometer. Folic acid (10 mg/ml) was used as a reference for this experiment. The percent loss of Folic acid in the samples was calculated based on the radical scavenging activity calculations from the absorbance value of DPPH assay as seen in Table 2.

As seen in Table 2 the amount of folic acid in broccoli goes down as the internal temperature of broccoli increases resulting in increased readings of DPPH in the assay. Initially as cooking resumes, the internal temperature of broccoli is raised and the amount of detectable folic acid increases which could be attributable to access of folic acid owing to breakdown of cellulose. However, as the temperature increases the folic acid gets denatured as evident in table 2. The results emphasize on the importance of regulating the cooking time such that the internal temperature of vegetables is not raised beyond a certain value (not-to exceed temperature).

TABLE 2
Effect of increase in internal temperature
of Broccoli on the % loss of Folic acid

| Time of cooking | Internal temperature | % loss of Folic acid |
|---|---|---|
| 0 min | 18.3° C. | 0% |
| 4 min | 22.5° C. | 1.9% |
| 8 min | 29.2° C. | 2.30% |
| 12 min | 68.6° C. | 43% |
| 16 min | 69.6° C. | 54.30% |

Based on this assay, the internal temperature range of (18-30° C.) seems conducive for cooking broccoli to prevent loss of Vitamin B and hence could be a not-to-exceed temperature for broccoli, During this temperature range the reduction in vitamin content seems to be down by 2-3%. This seems to be the not-to-exceed temperature for broccoli to preserve the vitamin B content. Cooking for a longer time than the not-to-exceed temperature range brings down the vitamin B content drastically (43% loss).

Vitamin C has ascorbic acid which is an important vitamin to strengthen one's immune system. Vegetables are a natural source of vitamin C. Vitamin C content is present in a high amount in certain vegetables such as bell pepper, onion, broccoli, radish, and zucchini.

Levels of Vitamin C (Ascorbic acid) can be tested using redox titration using iodine. As iodine is added during the titration, ascorbic acid gets oxidized to dehydroascorbic acid and the iodine is reduced to iodide ions. The iodine gets reduced to iodide as long as ascorbic acid is present. Once all ascorbic acid has been oxidized, the excess iodine reacts with the starch indicator to form blue-black starch—iodine complex which is the endpoint of titration.

Ascorbic acid content was evaluated in various vegetables by redox titration using iodine. Cooking of these vegetables showed a steady decline in the amount of vitamin C owing to the increase in the internal temperature of these vegetables as seen in Table 3.

Table 3 (A-E). shows the percent loss of vitamin C content as internal temperature increases. Vitamin C depletion was tested using titration with iodate solution. This loss of vitamin C is evident in each vegetable sample that was tested. Each vegetable had a different and unique internal temperature value (not-to-exceed) temperature above which there was reduction in vitamin C level

The change in titre values of iodate solution as the vitamin C gets depleted with increasing temperature were recorded for each vegetable sample. Percent loss of vitamin C is calculated using the following formula:

Grams of Ascorbic Acid: V1=volume (mL) I₂ required to change color of vegetable sample VC=volume (mL) I₂ required to change color of control, MC=# of Moles of C₆H₈O₆ in control sample, MM=Molar Mass of C₆H₈O₆

$$Ascorbic acid \{ g \} = \{ V 1 * MC / VC \} * MM$$

Percent Loss of Ascorbic Acid: VCR=vitamin C in raw vegetable sample, VCS=vitamin C in steamed vegetable sample

$$\% of Vitamin C lost = ( VCR - VCS ) / VCR ) * 100$$

Table 3. (A-E) Effect of increase in internal temperature of Broccoli on the % loss of Ascorbic acid

TABLE 3A
Data from BellPepper
BellPepper

| Internal Temperature | % loss of Ascorbic acid |
|---|---|
| 18.9° C. | 0.00% |
| 23.1° C. | 2.71% |
| 33.7° C. | 44.71% |
| 67.7° C. | 50.59% |
| 79.5° C. | 60.00% |

TABLE 3B
Data from Broccoli
Broccoli

| Internal Temperature | % loss of Ascorbic acid |
|---|---|
| 18.3° C. | 0.00% |
| 22.5° C. | 2.13% |
| 29.2° C. | 41.94% |
| 68.6° C. | 54.84% |
| 79.6° C. | 67.74% |

TABLE 3C
Data from Onion
Onion

| Internal Temperature | % loss of Ascorbic acid |
|---|---|
| 19.5° C. | 0.00% |
| 24.6° C. | 3.85% |
| 34.6° C. | 47.31% |
| 69.8° C. | 52.31% |
| 71.5° C. | 60.38% |

TABLE 3D
Data from Radish
Radish

| Internal Temperature | % loss of Ascorbic acid |
|---|---|
| 16.3° C. | 0.00% |
| 26.4° C. | 1.29% |
| 35.1° C. | 28.57% |
| 69.5° C. | 42.86% |
| 72.5° C. | 57.14% |

TABLE 3E
Data from Zucchini
Zucchini

| Internal Temperature | % loss of Ascorbic acid |
|---|---|
| 19.8° C. | 0.00% |
| 27.8° C. | 2.2% |
| 35.3° C. | 34.29% |
| 71.6° C. | 42.86% |
| 78.5° C. | 57.14% |

Based on the data from Table 3. the internal temperature range of (18-30° C.) seems to be the not-to-exceed temperature range and is more conducive for cooking to prevent loss of vitamin C. During this temperature range the reduction in vitamin content seems to be down by 2-5%. Cooking for a longer time than the not-to-exceed temperature range brings down the vitamin C content drastically. Each vegetable has a not-to-exceed temperature range to preserve the vitamin content.

Another indicator of optimal cooking time of the vegetable is the denaturation of enzymes present in the vegetables. Catalase enzyme is one of the most abundant enzymatic antioxidant present in plants, that attenuates the levels of reactive oxygen species. These reactive oxygen species are known to accompany pathological disorders such as aging, cataract, cancer, nutritional deficiency, atherosclerosis, and diabetes. Enzyme assays capture the integrity of the protein structure of enzymes that are instrumental in determining the functionality of the enzyme.

Catalase enzyme assay comprising of hydrogen peroxide and UV spectrophotometric method was utilized to measure the enzymatic activity of catalase (and hence functional integrity of the enzyme) in cooked broccoli and onion samples. Catalase activity was expressed in units wherein one unit of catalase represents a 0.01 decrease in absorbance in one min) as seen in Table 4.(A,B).

TABLE 4A
Data from Broccoli
Broccoli

| Time of cooking | Internal temp | Catalase/min/mg |
|---|---|---|
| 0 min | 18.3° C. | 35.4 |
| 4 min | 22.5° C. | 36.7 |
| 8 min | 29.2° C. | 31.5 |
| 12 min | 68.6° C. | 17.5 |
| 16 min | 79.6° C. | 9.6 |

TABLE 4B
Data from Onion
Onion

| Time of cooking | Internal temp | Catalase/min/mg |
|---|---|---|
| 0 min | 19.5° C. | 40.2 |
| 4 min | 24.6° C. | 42.3 |
| 8 min | 34.6° C. | 37.3 |
| 12 min | 69.8° C. | 14.5 |
| 16 min | 71.5° C. | 3.8 |

The internal temperature range of (18-25° C.) of the vegetables showed to be more conducive for cooking of vegetables to prevent denaturing of enzyme catalase (Table 4A,B). The enzyme activity during the temperature range seems to be reduced by about 4-8% in the vegetables. After that temperature, the enzyme activity reduces drastically and majority of the enzyme is denatured.

An internal temperature range for each of the vegetable needs to be regulated so as not to destroy the essential micronutrients and enzymes in the food when they are overcooked. These values for each vegetable can be predetermined.

A database of internal temperature of various vegetables corresponding to the change in the nutrient content (various vitamins and micronutrients) of each vegetable is documented (not shown). As indicated earlier, the internal temperature of the cooking pot is slightly higher than the internal temperature of vegetables (3-6 degrees) during the initial time of cooking vegetables. As cooking progresses, the internal temperature of the cooking pot matches the internal temperature of the vegetable. Therefore the database with the optimal temperature for cooking the vegetable can be correlated with the internal temperature of the cooking pot.

For example, at 4 min of cooking broccoli, the internal temperature of broccoli was 24.6° C. and the internal temperature of the cooking pot was 27° C. At 8 min of cooking time, the internal temperature of the cooking pot and broccoli was 29.2° C.

A cooking device that can capture changes in the internal temperature of the pot can indicate the internal temperature of the vegetables and hence enable the use of a method of cooking where the food is cooked to a temperature to maintain the optimal amount of nutritional content and keep the food easy to digest by a slight breakdown of the cell walls.

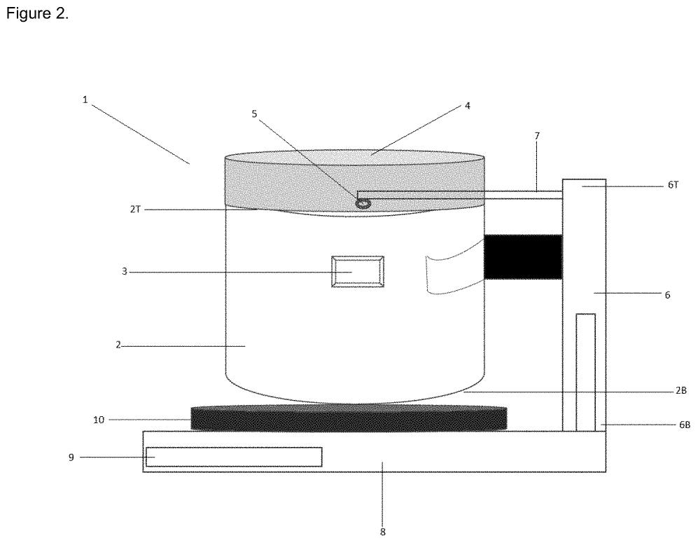

FIG. 2 is an embodiment of an automated cooking device unit 1 monitoring the internal temperature of the cooking pot and regulating the cooking of food. The cooking device unit 1 houses a cooking pot 2 preferably made up of stainless steel, anticorrosive material or any material that is resistant to increased temperature and corrosion. The cooking pot 2 has a pot holder 3 to lift the pot. Cooking pot 2 has a top 2T and a bottom 2B.

The top 2T of the cooking pot can be fitted with a cooking lid 4. The cooking lid 4 is fitted with an temperature sensor 5, for example an infrared temperature sensor. Temperature sensor 5 can monitor the change in internal temperature of the cooking pot 2.

Automated cooking device unit 1 has a main control circuitry housing 6. Main control circuitry housing 6 has a top 6T and a bottom 6B. The temperature sensor 5 is connected to the top 6T of the main control circuitry housing 6 by a electric cable connector 7. The electric cable connector 7 conveys the information collected by the infrared sensor to the main control circuitry housing 6.

The bottom 6B of the main control circuitry housing 6 is connected to a base support plate 8. The base support plate S includes a heater controller 9 that receives signal from the main control circuitry housing 6.

The main control circuitry housing is connected to the database of information on the optimal temperature for cooking of each vegetable.

A heating device 10 is housed on the base support plate and connected to the heater controller 9. The heating device 10 may be electric coil or ceramic based supplying direct heat or induction heating to the cooking pot 2.

The heating device 10 can be turned off based on the signal received from the main control circuitry housing 6. The main control circuitry housing 6 sends a signal to the heating device 10 based upon the information in the database for the optimal cooking temperature. Based on the signal the heating device can be regulated and can be turned off.

Another embodiment of the cooking device unit comprises a smart unit that enables selecting the programs for cooking the certain type of vegetable or vegetable mixture. The device comprises a processor and memory for storing not-to-exceed temperatures for various vegetables. The preferred device comprises a lid with a temperature sensor mounted to the lid. The temperature sensor is configured to monitor the internal temperature of a cooking pot. An output device is preferably connected to the temperature sensor for displaying temperature changes.

The cooking device preferably also comprises an automated cooking device unit. The automated cooking device controls a heating unit configured to heat the contents of the cooking pot. The heating unit can be housed in a base support plate comprising a heating controller, which is connected to the main control circuitry housing. The rain control circuitry housing can be connected to the temperature sensor housed in the lid by an electric cable carrier.

A microprocessor coupled to a database memory of the optimal cooking temperature of each vegetable can be programmed to control the heating unit.

The selected program regulates the cooking time and temperature of the food being cooked. The smart unit is connected to the computer or processor based microcontroller PCB housed in the main control circuitry housing. A control panel in the assembly housed in the main control circuitry housing controls the heating time and intensity of the device. The control panel is connected to a memory assembly that has programs related to different types of vegetables and their cooking temperature values.

A preferred embodiment of the cooking lid fitted with a temperature sensor such as an infra-red sensor may be connected to an output device to display the temperature change. The output device may be in the form of an alarm, a light, or any other means of display to indicate the temperature change.

Another embodiment of the cooking lid comprises an artificial intelligence system that can monitor the morphological changes of the vegetable structure and connect to the regulation of cooking time.

Another embodiment of the invention comprises of a cooking pot with a fitted temperature calculating device that can display the internal temperature of the vegetables.

Another embodiment of the invention comprises of the cooking pot with a lid assembly that can have a point of contact with the vegetables being cooked and capture the change in morphology of the vegetables by monitoring the texture change.

Another embodiment of the invention comprises of a cooking pot with a temperature resistant microscopic device attached to it. The image can be viewed on a smart phone to detect the amount of vegetable cooked.

An alternative embodiment of the invention comprises of a method to determine the nutrient content of the cooked vegetables in real time using enzyme assay.

Another embodiment of the invention comprises of a method to determine the reduction of vitamin level in the food as it is getting cooked using vitamin assays.

Another embodiment of the invention comprises of a method of calculating the micronutrient content in the cooking vegetables using quantitative assays.

Another embodiment of the invention comprises of a method of calculating the micronutrient content in the cooking vegetables using qualitative assays.

The present disclosure is to be considered as an exemplification of the invention, and is not intended to limit the invention to the specific embodiments described above. Various modifications may be made thereto without departing from the spirit of the invention as claimed.

What is claimed is:

1. A method of cooking a vegetable to preserve the nutritional content comprising the steps of:
selecting a first vegetable to cook,
determining a not-to-exceed temperature for the vegetable, the not-to-exceed temperature being a temperature that has been predetermined to be the lowest temperature that will break down a cell wall of the vegetable;
placing the vegetable to be cooked in a cooking pot,
adding liquid to the cooking pot,
increasing the internal temperature of the cooking pot by applying heat,
monitoring the internal temperature of the cooking pot, and
removing the heat prior to or when the internal temperature of the cooking pot reaches the not-to-exceed value.

2. A method of finding a not-to exceed temperature for cooking a vegetable while maximizing nutrition comprising the steps of:
selecting a vegetable,
placing the vegetable in a cooking pot,
adding liquid to the vegetable,
increasing the internal temperature of the cooking pot by applying heat,
removing a sample of the vegetable, noting the internal temperature, and observing the percentage of cells with broken cell walls,
if the percentage of cells with broken cell walls is less than one percent, continue cooking,
if the percentage of cells with broken cell walk is between 1 and 10%, documenting the internal temperature at the removal step as the not-to-exceed temperature of the vegetable.

3. A method of finding a not-to exceed temperature for cooking a vegetable while maximizing nutrition comprising the steps of:
selecting a vegetable,
placing the vegetable in a cooking pot,
adding liquid to the vegetable,
increasing the internal temperature of the cooking pot by applying heat,
removing a sample of the vegetable, noting the internal temperature, and running a vitamin assay,
if the percentage of vitamin loss is less than two percent, continue cooking,
if the percentage of vitamin loss is between 2-5%, documenting the internal temperature at the removal step as the not-to-exceed temperature of the vegetable.

4. A method of finding a not-to exceed temperature for cooking a vegetable while maximizing nutrition comprising the steps of:
selecting a vegetable,
placing the vegetable in a cooking pot,
adding liquid to the vegetable,
increasing the internal temperature of the cooking pot by applying heat,
removing a sample of the vegetable, noting the internal temperature, and running a enzyme assay,
if the percentage of enzyme activity loss is less than five percent, continue cooking,
if the percentage of enzyme activity loss is between 4-8%, documenting the internal temperature at the removal step as the not-to-exceed temperature of the vegetable.

5. A cooking device for cooking a vegetable to preserve the nutritional content comprising
a lid,
an temperature sensor mounted to the lid for monitor the internal temperature of a cooking pot;
said temperature sensor connected to an output device that displays the temperature change.

6. The cooking device of claim 5 further comprising
an automated cooking device unit, the automated cooking device unit housing a cooking pot supported by a heating unit configured to heat the contents of the cooking pot,
the heating unit housed on a base support plate comprising of the heater controller,
the base support plate connected to the main control circuitry housing,
the main control circuitry housing connected to the temperature sensor housed in the lid by an electric cable carrier,
the main control circuitry housing connected to a microprocessor coupled to a database memory of the optimal cooking temperature of each vegetable,
the microprocessor is programmed to control the heating unit.

7. The cooking device unit of claim 6 connected to a SMART system.
